(19) United States
(12) Patent Application Publication
(10) Pub. No.: US 2014/0107683 A1
(43) Pub. Date: Apr. 17, 2014

(54) Fluid-Surgical Instrument With Variable Spray Image

(71) Applicant: ERBE Elektromedizin GmbH, Tubingen (DE)

(72) Inventors: Ralf Kühner, Stuttgart (DE); Lothar Mitzlaff, Lagos (PT)

(73) Assignee: ERBE Elektromedizin GmbH, Tubingen (DE)

(21) Appl. No.: 14/042,059

(22) Filed: Sep. 30, 2013

(30) Foreign Application Priority Data
Oct. 11, 2012 (EP) 12188164.3

(51) Int. Cl. A61B 17/3203; A61B 17/32

(52) U.S. Cl. CPC A61B 17/32037; A61B 17/320068
USPC 606/169

(57) ABSTRACT

A fluid-surgical instrument includes a nozzle (20) to which a vibration can be applied. This concept can be used for changing the direction of the jet during the operation of the nozzle to generate an artificial wiping motion or to impart the nozzle with only micromovements that inject into the fluid jet a transverse pulse and thus lead to a change of the jet image. It is possible to combine both effects with each other. In this manner, the surgeon can retrieve, on command, e.g., different jet shapes made available by software technology and generate such jet shapes with one and the same instrument. In doing so, the surgeon can achieve varying effects without having to change the instrument and the nozzle.

### RELATED APPLICATION(S)

This application claims the benefit of European Patent Application No. 12188164.3 filed Oct. 11, 2012, the contents of which are incorporated herein by reference as if fully rewritten herein.

### TECHNICAL FIELD

The invention relates to a fluid-surgical instrument for the surgical treatment of tissue.

### BACKGROUND

In water jet surgery, a water jet is used for severing or rinsing away or dissolving tissue. To accomplish this, a water jet surgical instrument is used to apply water jets exiting from the distal end of said instrument to the biological tissue. Depending on the kinetic energy and the shape of the generated jet, it is possible to achieve different tissue effects.

In particular in endoscopic instruments, the nozzle and its exit opening are very small. However, even the smallest geometric deviations in a nozzle can lastingly affect the desired jet shape. In doing so, the form of the water jet is directly related to the geometric configuration of the nozzle. Therefore, it is a logistical problem to provide various nozzle shapes for generating different jet shapes and treatment effects. In particular, in the case of one-way devices that are designed for a single use only, the frequently great effort needed for manufacturing specific nozzles is not justified. In addition, a nozzle change or instrument changes is necessary if the surgeon desires another jet shape.

Examples of water jet surgery can be learned from Japanese utility model JP 60-192808, and from documents German DE 34 21 390, DE 30 19 115 as well as DE 37 15 418 A1. These show various nozzle shapes as examples in FIGS. 30, 31, 32, these being used as desired.

In particular in the treatment of parenchymal tissue, for example when dissolving such tissue, it is frequently desired to spare structures such as blood vessels, nerves, connective tissue or the like that are to be maintained. Regarding this, it has been known, for example, to work with a fan-shaped jet, the fluid droplets of which display relatively little energy by themselves, so that soft tissue is dissolved, however, firmer tissue is not attacked. On the other hand, at times tissue-severing or tissue-cutting functions are needed, in which case a sharply bundled jet acts on the tissue.

In order to generate these different jet shapes, it is common to use different nozzles. To do so, surgeons need to change instruments.

### SUMMARY

It is an object of the invention to provide a concept with which the various effects in the fluid-surgical treatment of tissue can be implemented with minimal efforts and the greatest possible reduction of operating time.

The fluid-surgical instrument in accordance with the invention may be an instrument that can be used for open surgery or for endoscopic procedures. In any event, it comprises at least one fluid conductor that feeds fluid—preferably NaCl solution—to a nozzle. In doing so, the fluid conductor extends through a carrier that is represented, for example, by a handle to be inserted into an instrument to be used in open surgery, or by an elongated element, e.g., a hose, or a tube. The hose or the tube may be inserted as a probe through the endoscope into a body or may itself represent an endoscope.

The nozzle is arranged so as to be movable relative to the carrier in order to perform an oscillating motion. In doing so, one or more actuators are disposed to move the nozzles in a specifically oscillating manner. In doing so, the nozzle may oscillate in one or two directions—transversely to the jet direction. Additionally or alternatively, the nozzle may be imparted with a pivoting motion about one or two axes positioned transversely to the jet direction. It is possible to impart the nozzle with a wobbling motion.

The actuator that allows the nozzle movement may itself be part of the instrument. In this case, the actuator is non-detachably connected to the instrument. It is also possible for the actuator to form a unit that is separate from the instrument, said unit being designed so as to be detachable. The actuator unit may then meet the requirements of a reprocessable unit.

Each of said measures allows a variation of the jet image of the linear jet exiting from the nozzle. This image may represent a linear, strip-shaped area, a circular area, a polygonal area or an area that is rounded or has corners or is ring-shaped.

Depending on the amplitude and frequency of the nozzle movement, at least two operating modes can be achieved:

In a first mode, the nozzle is pivoted within an angular range that has the size of the region to be swept over by the jet. This corresponds to an artificial tremor or a wiping motion that is otherwise performed by the surgeon in order to perform large-area tissue ablations, for example.

In a second mode, the nozzle is pivoted within an angular range or moved linearly in lateral direction, this range being clearly smaller than the region to be swept over by the jet. In doing so, the nozzle is fast enough for the jet to be superimposed by an oscillating transverse pulse in addition to the pulse directed in jet direction. Together with the longitudinal pulse directed in exit direction, the jet shape is determined by the frequency and size of the transverse pulse. In this manner, one or the same nozzle can be used to achieve different jet shapes, e.g., a straight cutting jet when the nozzle is non-oscillating, a fan jet when the nozzle oscillates linearly or pivotally in a transverse direction, a conical hollow jet when the nozzle is imparted with an oscillating circulatory motion, as well as a conical solid jet when the nozzle is imparted with pulses of alternating sizes and acting in transverse direction by appropriate triggering signals of actuators acting in transverse direction.

In the first mode, the nozzle oscillates at low frequency and high amplitude. In the second mode, the nozzle oscillates at high frequency and low amplitude. Mixed forms of the first and second modes are possible at medium frequency and medium amplitude. It is also possible to superimpose the first and the second modes. One examples of this is when the nozzle is used to generate a pivoting fan jet. All the other aforementioned shapes of the first and second modes may be superimposed.

The carrier that extends through the fluid conductor may be a handle, as mentioned, or a rigid tube, or also a hose displaying a certain flexibility, that is inserted, e.g., though an endoscope, into a body cavity. A nozzle is arranged on the distal end of this carrier. At least one actuator is effective between the carrier and the nozzle, so that the nozzle can be moved in a controlled manner relative to that carrier. To accomplish this, the nozzle may be elastically supported, e.g., elastically, by the carrier. The actuator itself may act as a support.

The actuators provided for the oscillating motion of the nozzle may be piezo actuators, vibramotors or the like. With them, it is possible to superimpose the motions of the water jet triggered by the surgeon by means of micromotions or microvibrations. Consequently, one and the same geometric configuration of the nozzle that is given by a cylindrical bore, for example, can be used to create or “form” different shapes of the applied water jet almost as freely as desired.

As a result of the invention it is possible to produce the most varied shapes of jets with one and the same nozzle. The otherwise existing fixed relationship of the jet shape with the geometric configuration of the nozzle is eliminated. It is also possible to produce more complex jet shapes than is possible with simple nozzles that are otherwise only suitable for generating a cutting, i.e., thin, laminar jet with high kinetic energy. In doing so, the expense and effort required for providing surgical instruments is reduced as is the time required for surgery when different jet shapes become necessary during an operation. In particular, the invention is suitable when one-way instruments are used, the jet shape of said instruments being defined by—in addition to the applied fluid pressure—only the actuation signals that are additionally applied to the actuator.

The possibility of using the simplest geometric nozzle configurations allows very small nozzle designs even of materials that are hard to machine such as, e.g., high-temperature resistant materials, e.g., tungsten. In doing so, the nozzle can be used not only as the fluid-surgical component but, at the same time, as the electrode for electrosurgical applications. This, too, contributes to a shortening of the operating times and the improvement of the quality of the surgical treatment.

The instrument is preferably associated with a control device for the actuator in order to appropriately feed the actuator with triggering signals that can preferably be switched by means of a selecting device in order to achieve different treatment effects in that various jet images are formed. The control arrangement may be part of a supplying medical device. Alternatively, the control device may be built into the instrument.

In the simplest case, the nozzle can be imparted with circular motions by means of the actuator, said motions generating a cylindrical or conical jet with different diameters. A strictly translatory vibration, preferably perpendicularly to the direction of flow (or also oriented in another angle) may produce an area-covering, fan-shaped jet.

It is possible to impart the distally arranged nozzle or a distal section of the instrument on another appropriate probe or an applicator with vibrations producing the desired jet shape or to also impart a longer shaft part or also the handle of an instrument itself with vibrations, in which case the shaft transmits the vibrations to the distal section. It is possible to combine both measures.

A selection device may be provided for the adjustment of a desired jet shape, said selection device comprising one or more control elements. One or more such control elements may be arranged directly on the handle of the surgical instrument or a supplying medical device that provides the pressurized fluid, for example, on a foot switch.

Preferably, the various jet shapes or operating modes are associated with corresponding triggering signals that are defined by data stored in a memory and that can be retrieved as needed. Such predefined programs or data facilitate the use. However, it is also possible to provide, e.g., the frequency amplitude and curve shape of the triggering signals in a freely selectable manner within limits, so that the surgeon can adjust the jet shape as desired, without being bound to the predefined jet shapes. For example, a high oscillation frequency of the nozzle can convert a hard jet into a fan jet that—in the close-up range of the nozzle, has the same effect, i.e., the same tissue effect—as a linear, i.e., hard jet, and thus has a cutting effect. Due to the loss of kinetic energy occurring with increasing distance from the nozzle, however, said jet becomes softer with increasing nozzle distance, as a result of which it can be absorbed readily by the tissue and deeper tissue layers and thus will no longer cause any perforation or at least offers greater safety against perforation.

The oscillation of the nozzle may also be used to superimpose a wiping motion onto the jet. For example, this can facilitate rinsing away parenchymal structures by means of the water jet in order to expose blood vessels, nerves and tumors. In this manner, the water jet does not act too deeply and thus does not cause any damage to deeper-lying non-visible tissue structures. The sweeping motion that is usually to be manually performed can be performed by oscillating the nozzle as described hereinabove. In doing so, an artificial tremor is generated, as it were. Ideally, the actual handle is mechanically uncoupled from the application part in such a manner that no disruptive vibrations are transmitted to the user, i.e., the surgeon. Again, the intensity, shape and direction of these transverse movements of the nozzle can be adjusted on the supplying device and/or the instrument and or on another control part such as, for example, a pedal.

In order to generate specific jet shapes, it is possible to combine the sweeping motion of the nozzles displaying preferably a lower frequency, however a greater amplitude, with the oscillating motion of the nozzle displaying preferably a higher frequency. To accomplish this, one or more actuators can be used.

As mentioned, the fluid-surgical instrument is suitable for the gentle parenchymal dissolution, for the exposure of tumors and for the treatment of wound healing dysfunctions in cases where large-surface areas must be treated. The combination of the control of the jet shape and the control of the sweeping motion make possible a uniform treatment of large-area tissue regions and, compared with a laminate jet that must be manually guided, significantly facilitates the performance of an operation.

In addition, the water jet can be pulsed. This can be accomplished by introducing pressure oscillations in the fluid channel or fluid conductor. The pressure oscillations can be generated on the proximal end of the fluid channel. Preferably, however, they can be generated by means of a piezo element, e.g., on the distal end in the vicinity of the nozzle, so that individual droplets of the severing medium impact the tissue at very high kinetic energy. In doing so, it is possible to use small amounts of fluid to achieve a relatively large ablation effect or a strong effect on the tissue. In addition, e.g., one or more actuators acting as valves may be provided on the nozzle in order to actively interrupt the fluid jet and thus influence the tissue effect.

In particular in endoscopic applications, the carrier may comprise additional elements such as photoconductors, a lens system, RF-surgical means, e.g., at least one electrode and at least one electrical line, an evacuation channel and the like.

### BRIEF DESCRIPTION OF THE DRAWINGS

Additional details of advantageous embodiments of the invention can be learned from the description and the drawings. They show in

FIG. 1 a fluid-surgical instrument in the treatment of an organ and a supplying device for the open surgical treatment, each in extremely schematized representation;

FIGS. 2 and 3 exemplary embodiments of the fluid-surgical instrument as in FIG. 1, in a schematized basic representation;

FIG. 4 a fluid-surgical instrument for endoscopic surgery, in a schematized representation;

FIG. 5 an exemplary embodiment of a fluid-surgical instrument for the laparoscopic use, in a schematized sectional longitudinal representation;

FIG. 6 a modified embodiment of the instrument as in FIG. 5, in a schematized sectional longitudinal representation;

FIG. 7 the instrument as in FIG. 6, in a schematized front view;

FIG. 8 triggering signals for the actuators of the instrument as in FIG. 7, for the generation of a conical solid jet; and

FIG. 9 size and orientation of the transverse pulse of the jet during the generation of a solid cone, as a diagram.

### DETAILED DESCRIPTION

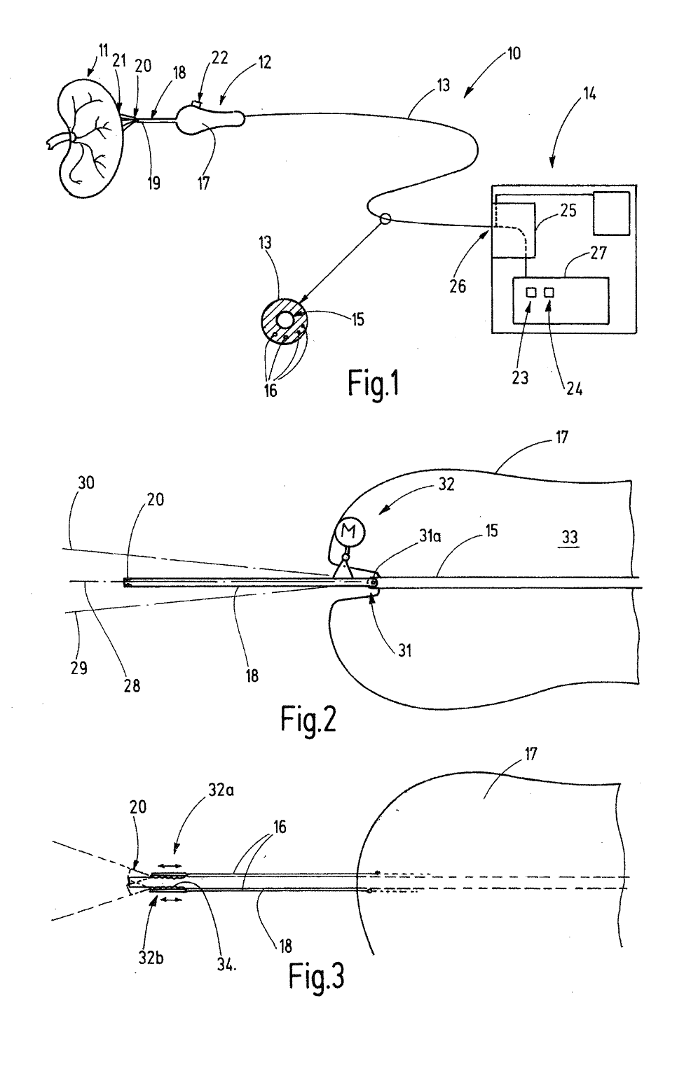

The system 10 for the fluid-surgical treatment of an organ 11 or another biological tissue comprises a fluid-surgical instrument 12 that is connected to a surgical feed device 14 by way of a line 13. As shown in FIG. 1 in cross-section, the line 13 comprises at least one fluid conductor or fluid channel 15 that feeds pressurized fluid, e.g., water (NaCl solution) to the instrument 12. In addition, the line comprises preferably at least one or several electrical lines 16 that are insulated from each other and are disposed to transmit the electrical power from the device 14 to the instrument 12. Said device also comprises an applicator 18 extending from said device's handle 17, in which case a nozzle 20 is provided on the distal end 19 of said applicator. This nozzle allows a fluid jet 19 to exit, said jet being shown in FIG. 1 as the exemplary form of a fan jet or conical jet.

The instrument 12 comprises at least one control element 22 that can affect, for example, the shape of the jet 21. The control element 22 can be arranged on a handle 17, as illustrated. Alternatively, the activation of the instrument 12, as well as the shape and the intensity of the jet 21, can also be achieved with a control element that has the form of a foot switch. Additionally or alternatively, it is possible to provide one or more control elements 23, 24 on the device 14, said control elements being suitable for controlling the properties of the jet 21, in particular its shape. To accomplish this, the device 14 comprises an arrangement 25, for example in the form of a pump, by means of which the nozzle 20 can be supplied via the applicator 18 and the line 13—there, in particular, via the fluid channel 15—with the pressurized fluid. To do so, the proximal end 26 of the line 13 is connected to the arrangement 25. The electrical lines 16 are connected to a control arrangement 27, by means of which control pulses can be generated, said pulses reaching one or several actuators via the lines 16, said actuators being used to move the nozzle 20 in transverse direction. Alternatively, it is also possible to arrange the control arrangement directly in the instrument 12 and to supply it with electrical power via the lines 16. And RF generator 38 may be provided for applying the nozzle and/or the applicator 18 with RF power via one of the lines 16 for performing RF-surgical operations.

FIG. 2 is a schematic of a design comprising a movable nozzle 20. In that case, the nozzle 20 can be moved out of its center position into different pivot positions. In doing so, the entire applicator 18 is pivoted, e.g., in order to achieve an artificial wiping motion. The various longitudinal directions associated with the respective pivot positions of the applicator 18 are symbolized by the chain lines 28, 29, 30. The pivot angle may be relatively large and, e.g., correspond to the sweeping fan.

The applicator 18 may be connected to the remaining fluid channel 15 via a pivotable connection 31. This connection 31 may be, e.g., an elastic connection or the like.

The tubular applicator 18 is connected to an actuator 32 that is disposed to move the applicator 18 and thus ultimately the nozzle 20 in an oscillating motion transversely to the longitudinal directions 28 and 29, 30, respectively. In doing so, the actuator 32 is carried by the handle 17 that, in this case, represents a carrier 33 through which extends the fluid channel 15. The output of the actuator 32 acts laterally on the applicator 18. The actuator 32 may be a piezo drive, a magnetic drive, a vibramotor, a motor with eccentric drive for the applicator 18, or the like.

Whereas the actuator 32 in the embodiment of FIG. 2 pivots the entire applicator 18, it may also be useful to only pivot a short part thereof or to move the nozzle 20 in transverse direction. FIG. 3 shows an exemplary embodiment in a highly schematic manner. In this case, the applicator 18 bears, on its end, the nozzle 20 whose elastic, pivotable coupling to the applicator 18 is symbolized by a bellows 34. Instead of a bellows, it is also possible to use other technically flexible elements, e.g., a hose or the like.

In order to impart the nozzle 20 with a transverse vibration, the bellows 34 or the otherwise movable element may be bridged by one or more actuators 32a, 32b—in this case, e.g., longitudinally contracting, piezo elements, whereby, in each case, one of their ends is connected to the nozzle 20 and the other end to the applicator 18. Again, the lines 16 are disposed to transmit triggering signals to the actuators 32a, 32b. The pivot angle or lateral path of movement of the nozzle 20 may be significantly smaller than the opening angle defined by the jet.

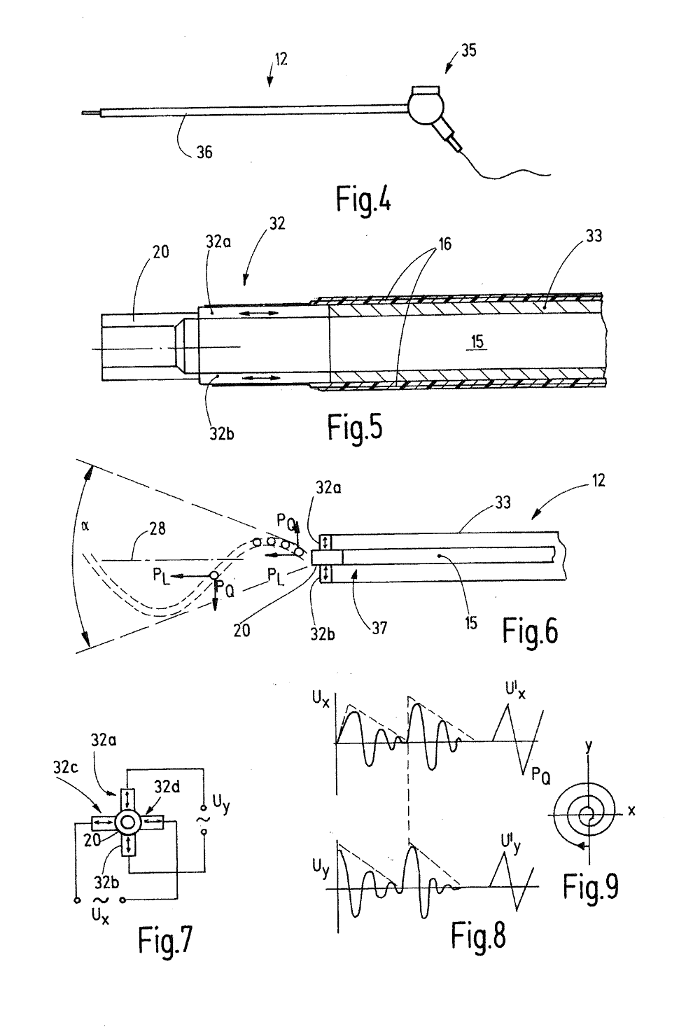

The instrument 12 may be configured in a similar manner if it is designed for the use in an endoscope 35. As schematically shown by FIG. 4, said endoscope comprises a tubular shaft 36 through which extends the instrument 12. FIG. 5 shows a corresponding, flexible carrier 33 that is configured as a flexible tube or hose here and that contains the fluid channel 15, as well as the lines 16. Again, the nozzle 20 is provided on the end. Preferably, the nozzle 20 is provided with a cylindrical bore in order to generate a laminar hard jet. The connection between the carrier 33 and the nozzle 20 again is accomplished by an actuator arrangement 32 that is associated with one or more actuators 32a, 32b. For example, these may be longitudinally operating piezo actuators that, e.g., are controlled in opposite directions in order to impart the nozzle 20 with a pivoting micro movement or a transverse movement.

FIG. 6 shows another embodiment of an instrument 12. This embodiment can be used as an instrument 12 for open surgery as in FIG. 1, and also as a laparoscopic instrument as in FIG. 4. The carrier 33 is configured, e.g., as a flexible hose 37 and contains the channel 15. The distal end of the hose 37 is connected to the nozzle 20. As is shown by FIG. 7, the latter is associated with several—e.g., four—actuators 32a, 32b, 32c, 32d that can impart the nozzle 20 with a motion directed transversely to the longitudinal axis. The actuators 32a-32d are configured, e.g., as piezo elements, whereby oppositely located piezo elements 32 and 32b or 32c and 32d are actuated by appropriate triggering signals Uₓ and U_{y} (U′ₓ and U′_{y}). By making available appropriate triggering signals Uₓ and U_{y} that have been adjusted to each other in view of frequency, waveform and phase, it is possible to obtain almost any desired jet shape.

FIG. 6 illustrates the embodiment of a fan jet. In this case only two of the oppositely arranged actuators 32a, 32b are activated in such a manner that the nozzle 20 oscillates transversely to the main exit direction and the longitudinal axis 28. In doing so, the transverse oscillations may display a relatively low amplitude. Decisive here is the rapidity of the vibration (i.e., the (maximum) velocity of the nozzle in transverse direction) that determines the pulse component directed transversely to the longitudinal direction 28. FIG. 6 illustrates the pulse components p_{L} for the longitudinal direction and p_{Q} for the transverse direction. As is obvious, the longitudinally directed pulse component p_{L} is essentially constant. However, the transversely directed pulse component p_{Q} only oscillates, i.e., it is a function of time. In this manner, a fan jet is produced that has an opening angle α that can be defined by the size and frequency of the amplitude of the triggering signal.

If the nozzle 20 is guided on a circle, e.g., by actuating the other actuator pair 32c, 32d, a hollow conical jet is formed accordingly. It is also possible to produce a solid conical jet when the triggering signals Uₓ and U_{y} that are offset relative to each other by 90 degrees regarding their oscillations do not have a constant amplitude but are provided imparted with a saw-tooth modulation. FIG. 9 shows the result. The transversely directed pulse p_{Q} is described by a circulating vector having a length that is periodically reduced from a maximum to zero.

The triggering signals Uₓ and U_{y} for the actuator pair 32a, 32b and/or 32b, 32c can be changed by means of the control elements 22, 23 and/or 24 in order to change the shape of the jet. For example, the Figure shows other curve forms U′ₓ and U′_{y} for generating the jet shapes. The curve forms U′ₓ and U′_{y} may differ from the curve forms Uₓ and U_{y} regarding various parameters such as curve form, amplitude, frequency among other things. Such parameters can be retrieved or adjusted by means of the control elements 23 and/or 24.

As is obvious, various signal forms can be used for adjusting various basic characteristics. If waveforms deviating from the sinusoidal form (rectangular vibration, saw-tooth vibration, triangular vibration, trapezoid vibration or the like) are applied to the piezo actors, fan jets operating in an edge-emphasized manner can comprise hollow conical jets that have a non-circular cross-section (e.g., square cross-section with corner-emphasized jet effect or other properties).

In accordance with the invention, a fluid-surgical instrument is provided, said instrument comprising a nozzle 20 to which a vibration can be applied. This basic concept can be used for changing the direction of the jet during the operation of the nozzle in order to generate an artificial wiping motion or in order to impart the nozzle with only micromovements that inject into the fluid jet a transverse pulse and thus lead to a change of the jet image. It is possible to combine both effects with each other. In this manner, the surgeon can retrieve, on command, e.g., different jet shapes made available by software technology and generate such jet shapes with one and the same instrument. In doing so, the surgeon can achieve varying effects without having to change the instrument and the nozzle.

### LIST OF REFERENCE SIGNS

10 System
11 Member
12 Instrument
13 Line
14 Device
15 Fluid channel, fluid conductor
16 Lines
17 Handle
18 Applicator
19 Distal end
20 Nozzle
21 Jet
22 Control element
23, 24 Control elements/selecting device
25 Arrangement
26 Proximal end of line 13
27 Control arrangement
28-30 Chain lines—longitudinal direction
31 Pivotable connection
32 Actuator 32a-32d
33 Carrier
34 Bellows
35 Endoscope
36 Tubular shaft
37 Hose
38 Generator

1. Fluid-surgical instrument (12) comprising:
a fluid conductor (15) that extends through a carrier (33) and to whose proximal end (26) a pressurized fluid can be applied,
a nozzle (20) that is connected to a distal end (19) of the fluid conductor (15) in order to allow a jet (21) of the fluid supplied by the fluid conductor (15) to the nozzle (20) to exit in an exit direction (28),
an actuator (32) that is in operative connection with the nozzle (20) in order to move said nozzle in an oscillating manner relative to the carrier (33).

2. Instrument as in claim 1 wherein the nozzle (20) is arranged on the distal end (26) of the carrier (33).

3. Instrument as in claim 2 wherein the actuator (32) is arranged between the nozzle (20) and the carrier (33).

4. Instrument as in claim 2 wherein the nozzle (20) is elastically supported on the carrier (33).

5. Instrument as in claim 1 wherein the actuator (32) is disposed to at least one of:
pivot the nozzle (20) about at least one axis oriented transversely with respect to the exit direction (28), or
move it along an axis that extends parallel to the exit direction (28).

6. Instrument as in claim 1 wherein the actuator (32) is disposed to at least one of:
pivot the nozzle (20) about two axes oriented transversely with respect to the exit direction (28), or
move it along one axis that extends parallel to the exit direction (28).

7. Instrument as in claim 1 wherein the actuator (32) comprises at least one piezo drive.

8. Instrument as in claim 1 wherein the actuator (32) comprises at least one vibramotor.

9. Instrument as in claim 1 wherein the actuator (32) is connected to a control arrangement (27) in order to supply to the actuator (32) triggering signals (Uₓ, U_{y}) generated by the control arrangement (27).

10. Instrument as in claim 9 wherein the control arrangement (27) is disposed to generate a periodic triggering signal (Uₓ, U_{y}).

11. Instrument as in claim 9 wherein the control arrangement (27) is disposed to generate at least two different triggering signals (Uₓ, U_{y}) and is provided with a selecting device (23, 24) controlled by the control arrangement (27) to switch from generating a triggering signal (Uₓ, U_{y}) to generating another triggering signal (U′ₓ, U′_{y}).

12. Instrument as in claim 11 wherein a first one of the triggering signals (Uₓ, U_{y}) is allocated to a first jet image and a second one of the triggering signals (U′ₓ, U′_{y}) is allocated to a second jet image.

13. Instrument as in claim 1 wherein the nozzle (20) is connected to an electrical line (16) that is connected to an RF-generator (38) in order to supply the nozzle with RF power.

14. Instrument as in claim 1 wherein an arrangement (25) for generating pressure pulses is located upstream of the nozzle (20).

15. Instrument as in claim 1 wherein the actuator (32) is a unit that can be separated from the instrument (12).

16. A method of operation for a fluid-surgical instrument (12), the method comprising:
passing fluid through a fluid conductor (15) that extends through a carrier (33) to a nozzle (20) that is connected to a distal end (19) of the fluid conductor (15),
moving the nozzle in an oscillating manner relative to the carrier (33) using an actuator (32) that is in operative connection with the nozzle (20).

17. The method of claim 16 wherein the moving the nozzle further comprises at least one of:
pivoting the nozzle (20) about at least one axis oriented transversely with respect to an exit direction (28) of the fluid through the nozzle, or
moving it along an axis that extends parallel to the exit direction (28).

18. The method of claim 16 further comprising the actuator receiving triggering signals (Uₓ, U_{y}) generated by a control arrangement (27).

19. The method of claim 18 wherein the receiving the triggering signals comprises:
receiving a first one of the triggering signals (Ux, Uy) and in response ejecting the fluid in a first jet image, and
receiving a second one of the triggering signals (U′x, U′y) and in response ejecting the fluid in a second jet image.

20. The method of claim 15 further comprising generating pressure pulses in the fluid.
